(19) United States
(12) Patent Application Publication
(10) Pub. No.: US 2004/0048317 A1
(43) Pub. Date: Mar. 11, 2004

(54) Means for diagnosing tumours

(76) Inventor: Elke Pogge Von Strandmann, Bochum (DE)

Correspondence Address: WILLIAM COLLARD, COLLARD & ROE, P.C., 1077 NORTHERN BOULEVARD, ROSLYN, NY, 11576, US

(21) Appl. No.: 10/362,843

(22) PCT Filed: Aug. 30, 2001
(86) PCT No.: PCT/EP01/10020

(30) Foreign Application Priority Data
Aug. 30, 2000 (DE) 100 42 827.4

(51) Int. Cl. C12Q 1/68; G01N 33/574; C07K 16/30

(52) U.S. Cl. 435/7.23; 435/6; 530/388.8

(57) ABSTRACT

The invention relates to a means for diagnosing tumours, especially malignant tumours in the human body and the animal body, using at least one substance which interacts with DCoH for in vitro diagnosis.

The invention relates to a means for diagnosing tumors, particularly malignant tumors in the human body and the animal body.

Malignant tumors comprise a heterogeneous group of various tumor types with regard to their biological behavior (e.g. invasiveness and tendency to metastasize) and their histogenetic origin. This heterogeneity is reflected in their different response to different forms of therapy. It is therefore essential to classify tumors as accurately as possible with regard to the aforementioned characteristics, and to assign them to these classifications. This is done, among other things, using so-called tumor markers. These are histological tissue changes and molecular cell components whose qualitative and quantitative characterization gives information about the presence, the type, and the progression of malignant diseases.

Known tumor markers include, for example, defined chromosomal aberrations, the ectopic or excessive formation of various hormones and, in particular, also the ectopic or excessive expression of different proteins.

The detection of an excessive gene expression on the mRNA level can take place, for example, by means of quantitative RT-PCR methods or Northern blot techniques, with RNA samples obtained from tumor material or serum. On the protein level, this takes place, for example, by means of immunohistochemical techniques, immune precipitation or Western blot methods using antibodies directed against the tumor markers. In this connection, the immunohistochemical characterization of tumor markers in tumor material taken from tissue sections has particular significance, since this method allows not only the detection of tumor markers but also the precise characterization of the dimensions of the tumor that was removed. This characterization is important for the determination of the tumor size, as a further indication of the degree of malignity of the tumor. In particular, however, it serves as a post-operative check to ensure that the tumor was completely removed, which is the deciding factor for the success of the operation.

The genetic instability of malignant tumors represents a big problem for tumor diagnosis. The loss of cell differentiation that accompanies tumor progression often leads to a complete or partial loss of the expression of characteristic tumor markers, so that these can only be used to a weak extent, or not at all, for identification of the tumor in corresponding detection methods. It is also a particular problem, in this connection, that the cells of a tumor, in spite of their clonal origin, lose their genetic homogeneity over the course of tumor progression, which has the result that during the post-operative check of tissue sections, only partial regions of the tumor can be characterized. This results in the risk that incomplete tumor removal will not be recognized, or that the tumor size will be incorrectly determined.

The invention is therefore based on the task of making available alternative possibilities for tumor diagnosis on the basis of alternative tumor markers with expression patterns that differ from those of known tumor markers. Furthermore, there is a need, to a particular degree, for the use of substances and means for tumor diagnosis that are suitable for achieving as complete and homogeneous a characterization as possible of removed tumors in histological tissue sections.

This task is accomplished, according to the invention, with the use of DCoH as an alternative tumor marker.

The invention therefore relates to the use of at least one substance that interacts with DCoH, for the diagnosis of tumors.

The enzyme DCoH is the dimerization co-factor of the HNF-1 homeodomain proteins (HNF-1α and HNF-1β). By means of this mechanism, DCoH is involved in the control of gene expression, see D. B. Mendel et al., Science 254 (1991; 1762). At the same time, the protein is known as pterin-4α-carbinolaminedehydratase PCD and is involved in tetrahydrobiopterine regeneration, see B. A. Citron et al., Proc. Natl. Sci. USA 89, 1992, 11891. In this connection, PCD intervenes in the biosynthesis of L-tyrosine from L-phenylalanine. PCD is involved in the latter cycle together with phenylalanine hydroxylase, and participates in the regeneration of (6R)5,6,7,8-tetrahydrobiopterine. Independent of this, however, DCoH/PCD is also found in the vertebrate egg, in the pigmented epithelium of the eye, in the skin, and in the brain, see E. Pogge v. Strandmann and G. U. Ryffel, Development 121 (1995), 1217; E. Pogge v. Strandmann et al., Int. J. Dev. Biol. 42 (1998), 53.

According to preliminary data concerning the purification and cloning of DCoH by D. B. Mendel et al., Science 254 (1991), 1762, both the peptide sequence and the nucleic acid sequence that codes for it was described, for the first time, for human, mouse, and rat DCoH, in U.S. Pat. Nos. 5,403,712 and 5,620,887. With regard to all the relevant structural data, reference is made to these publications. DCoH/PCD has proven to be a more or less universal principle in the development of vertebrates, particularly also for early development. It demonstrates both catalytic and regulating properties and is present in a large number of cell types. In some of these cell types, it is related to the nuclear transcription factors HNF-1α and HNF-1β, in others it is bound into the phenylalanine hydroxylase enzyme complex. The occurrence of DCoH/PCD in cell types in the absence of HNF-1 or phenylalanine hydroxylase indicates that the protein also interacts with cellular partners other than these.

The invention is based on experimental studies of the inventor, which showed that the cellular amount of DCoH, contrary to previous assumptions, is not related to the pigment status of cells. Surprisingly, in contrast, there is a relationship between the degeneration of cells—independent of their pigmentation status—and the increase in the cellular DCoH content. Thus, experiments showed that malignant melanomas demonstrate elevated levels of DCoH protein and mRNA, as compared with the surrounding, non-degenerate tissue and, in particular also non-degenerate melanocytes.

Furthermore, an increase in the cellular DCoH content is also found in tumors that are derived from cells that do not express DCoH at all in adults, or only to a slight degree. This is particularly surprising because normally, DCoH is expressed strictly specific to cell type.

Therefore DCoH is suitable as an alternative tumor marker.

The use, according to the invention, of substances that interact with DCoH in tumor diagnosis, expands the repertoire of tumor-specific markers that is available to a histopathologist. This expansion of the repertoire of special tumor antigens is significant in that only staining with different antibodies allows a reliable determination of the origin of the tumor according to an exclusion method. This is essential for the diagnosis as to whether the tumor being studied is a primary tumor or whether the tumor being studied is a metastasis of a tumor that comes from a different type of tissue.

Furthermore, the use according to the invention demonstrates the advantage, as compared with the state of the art, that it is possible to achieve a homogeneous characterization of tumors, using the stated substances, which tumors cannot be characterized at all using other markers, or can only be characterized in partial regions.

In this connection, the tumor diagnosis comprises both the identification and the imaging of tumors. The interaction between substance and DCoH relates to all of the interactions of the substances or parts of the substances with DCoH (protein or mRNA) or parts of DCoH in the intact or prepared tissue, intact or prepared cells or molecule mixtures, which are suitable for the detection of DCoH. Substances in the sense of the invention are compounds with a high or medium molecular weight, as well as mixtures containing these compounds. In this connection, proteins such as antibodies in the form of polyclonal antisera, monoclonal or recombinant antibodies, as well as peptides or nucleic acids are particularly suitable. The use, according to the invention, of the substances that interact with DCoH, can be based on any suitable method for the characterization and detection of DCoH expression. Conventional methods that yield information about the cellular protein or mRNA content of DCoH are particularly suitable for this purpose. However, methods for detecting the enzymatic activity of DCoH can also be used.

The use of the substances that interact with DCoH for diagnosing malignant melanomas represents a preferred exemplary embodiment of the invention, because DCoH expression is surprisingly greatly increased both in melanotic and in extensively de-differentiated, amelanotic malignant melanomas, as compared with non-degenerate tissue. The use according to the invention therefore allows a homogeneous and complete characterization of melanomas in tissue sections.

The expression of known melanoma-specific tumor markers is often lost with increasing differentiation, so that highly degenerate melanomas, in particular, cannot be characterized and identified, or can be characterized and identified only insufficiently. This holds true, for example, for the two melanoma markers that are frequently used for tumor diagnosis, gp100 and S100. The glycoprotein gp100 is specific for premelanosomas and is not expressed, or only very weakly expressed, in the particularly aggressive amelanotic malignant melanomas. As a result of this, a reliable identification and characterization is no longer possible using the monoclonal antibody HMB45, which is frequently used and directed against gp100.

For this reason, the use of antibodies against the protein S100, an acidic Ca-binding protein, is an additional aid in the identification and imaging of malignant melanomas, since its expression is independent of the melanotic status. However, this marker allows only an unsatisfactory characterization of malignant melanomas, since its immunohistochemical detection in tumor samples taken from tissue sections often allows only a characterization of partial regions of the tumor.

The use, according to the invention, of substances that interact with DCoH, therefore demonstrates the advantage, as compared with substances used for the detection of these markers, that it is possible to achieve a homogeneous and complete characterization, particularly of malignant melanomas.

Because of its suitability for a large number of analytical methods, the use of antibodies, according to the invention, is particularly practical. In this connection, practically any antibodies or mixtures of different antibodies can fundamentally be used, but the use of monoclonal antibodies is particularly advantageous. Specific purification of the antibodies using conventional techniques is also advantageous in order to reduce background signals.

According to a particularly preferred embodiment of the invention, at least one polyclonal antiserum that interacts with DCoH and/or at least one monoclonal antibody that interacts with DCoH are used for the diagnosis of melanomas.

The use of at least one nucleic acid that interacts with DCoH for diagnosing melanomas is just as practical. Possible nucleic acids are, for example, anti-sense nucleic acids or antisense oligonucleotides that hybridize with DCoH-mRNA or parts of it. These can be marked and detected using conventional techniques, and represent an alternative for immunohistochemical characterization of tumor tissue in histological sections. The use of DNA-oligonucleotides, which are used to detect DCoH as a PCR primer, within the scope of RT-PCR reactions, or as probes in RNA blot methods, is also suitable.

Furthermore, means for diagnosing tumors that contain at least one substance that interacts with DCoH are also an object of the invention. These means can consist of the substance itself or the substances themselves, or can have additional components. Liquid suspensions of the substances in suitably buffered media are practical. It is practical if the components serve to stabilize the substances (e.g. detergents, proteins, protease or nuclease inhibitors, etc.) or contribute to the detection method in some other way.

According to a preferred embodiment, the means according to the invention is used for diagnosing melanomas, whereby antibodies are particularly suitable as the substance contained in the means.

According to another preferred embodiment, the means according to the invention contains at least one nucleic acid that interacts with DCoH as the substance.

It is particularly practical to offer the substances that interact with DCoH, i.e. the means that contain them, in the form of commercially available kits that contain other substances and/or utensils that are necessary for the detection of DCoH. For example, PCR kits are practical, which contain not only the DNA primers that hybridize with DCoH, but also reverse transcriptase, a thermostable DNA polymerase, deoxynucleotides, and the necessary reaction buffers. The same holds true for kits for carrying out immunohistochemical staining methods, where it is practical if they contain not only the first antibody that interacts with DCoH, but also a marked second antibody in a stabilized buffer. Other types of forms of test kits, coordinated with the detection method, in each instance (Western blot, ELISA, etc.) also lie within the scope of the invention. The components necessary for this are generally known to a person skilled in the art.

The invention will be explained in greater detail below, using exemplary embodiments, the first of which is illustrated by FIG. 1, which relates to it.

FIG. 1 shows, as an example, a tissue section through a malignant melanoma, which was prepared in different ways.

FIG. 1 shows four different illustrations of differently prepared sections through a primary malignant melanoma that is embedded in paraffin.

The left top illustration of FIG. 1 shows the stemmed tumor of a nodular type, clearly circular in the center of the section, with many necrotic regions.

In the right top illustration of FIG. 1, the section was incubated with an antibody (produced according to Pogge von Strandmann et al. in 1995, produced analogously in 1998). The section was treated with the antibody before incubation, according to standard methods, in a commercially available microwave, in ProTaqsIV buffer from the company BIOCYC Luckenwalde, once for 6 min at 600 watts, after cooling, again at 600 watts to boiling, then at 180 watts for 5 min. The antibody of this batch was then diluted 1:400 in PBS pH 7.5, and the section was incubated for 32 min. In this illustration, a bridge antibody was used, which allows detection of the bound primary rabbit antibody. The detection of the bridge antibody took place by means of alkaline phosphatase bound to the second antibody. The color reaction took place using the Fast Red Naphthol System, according to standard methods. The stain is red; here, because of the black-and-white reproduction, it is gray. Counter-staining took place with hematoxylin.

In the left bottom illustration of FIG. 1, the subsequent section was not treated with the DCoH-specific antibody, but rather with commercially available antibodies against S100.

In the right bottom illustration of FIG. 1, the subsequent section was not treated with the DCoH-specific antibody, but rather with commercially available antibody HMB 45.

The S100 and HMB 45 stains involve the detection methods that are normally used in clinical diagnosis.

### EXAMPLE 2

Detection and Quantification of DCoH in Patient Serum

An enzymatic quick-detection method of an elevated DCoH concentration in the serum of patients is particularly simple. For this purpose, blood is first taken from patients who are suspected of having a malignant melanoma, and the serum is isolated from it by means of standard methods. The serum can be processed further direction, or can be temporarily stored at at least −20° C., preferably −70° C.

In this example, the DCoH detection takes place on the basis of an ELISA test (Enzyme Linked Immuno Sensitivity Assay), a conventional protein detection method. For the sake of better sensitivity, a so-called “sandwich” ELISA is preferably used. This test is based on two antibodies with a monoclonal or polyclonal origin, which detect different epitopes of the DCoH protein.

In the first step, the ELISA plate is coated with monoclonal mouse DCoH antibody (the dilution is dependent on the antibody, in each instance, and can be tested in advance by a person skilled in the art). After an intermediate blocking step of the free binding sites of the ELISA plate (usually by means of BSA or the like) and washing steps, incubation with the serum takes place, during which the interaction of the DCoH contained in the serum with the first DCoH-specific antibody found on the plate takes place. After additional washing steps, incubation with the second specific anti-DCoH antibody takes place (for example a polyclonal rabbit serum), and subsequently (again, after normal washing steps), incubation with a suitable “second” antibody takes place, which reacts specifically with the FC portion of the second anti-DCoH antibody. It is important in this technique that the first and the second specific anti-DCoH antibodies come from different species, in order to obtain binding of the “second” antibody only to the second anti-DCoH antibody.

Such species-specific second antibodies are commercially available and can be purchased coupled to various enzymes or fluorescent substances (e.g. to horseradish peroxidase (HRP) or intestinal calf phosphatase (CIP)). Depending on the type of enzyme coupled to the second antibody, a substrate—also commercially available—is subsequently applied to the ELISA plate in a suitable buffer, and incubated over a defined period of time, while reaction of the substrate takes place. In the present case, an HRP-coupled goat-anti-rabbit second antibody can be used as the second antibody (available, for example, from Pharmacia or Santa Cruz Technologies). Measurement and quantification of the generated signal takes place using suitable fluorometers.

Serum of healthy patients and, if necessary, additional negative samples (BSA, etc.) are treated in the same way. In this way, the “steady state” amount of DCoH protein contained in the serum can easily be determined from a comparison of the absolute and relative signal intensities of the patient serum samples with the negative controls. In this way, it can be quickly determined, without complicated biopsies and tissue removal, whether or not a malignant melanoma is present. Particularly subsequent to operative treatment of externally visible melanomas, tumor patients can thus be examined with regard to possible metastases, which are not externally visible, by means of blood samples repeated at intervals.

General protocols for carrying out the ELISA sandwich method are known to a person skilled in the art. The precise parameters must be tested for each specific antibody combination, which corresponds to standard laboratory methods. Suitable instructions are found in the relevant standard laboratory reference works.

1. Use of at least one substance that interacts with DCoH for the in vitro diagnosis of tumors.

2. Use according to claim 1, characterized in that the tumors are melanomas.

3. Use according to one of claims 1 or 2, characterized in that the substance contains at least one antibody.

4. Use of at least one polyclonal and/or monoclonal antibody that interacts with DCoH protein for the in vitro diagnosis of melanomas.

5. Use of at least one nucleic acid that interacts with DCoH for the in vitro diagnosis of melanomas.

6. Means for the diagnosis of tumors, containing at least one substance that interacts with DCoH.

7. Means according to claim 6, characterized in that the tumors are melanomas.

8. Means according to claim 6 or 7, characterized in that the substance contains at least one antibody that interacts with DCoH.

9. Means according to claim 6 or 7, characterized in that the substance contains at least one nucleic acid that interacts with DCoH.

10. Tumor diagnosis kit having at least one polyclonal and/or monoclonal antibody that interacts with DCoH.

11. Tumor diagnosis kit having at least one nucleic acid that interacts with DCoH.

12. Kit according to claim 10 or 11 for diagnosing melanomas.
